(19) United States
(12) Patent Application Publication
(10) Pub. No.: US 2026/0283247 A1
(43) Pub. Date: Sep. 24, 2026

(54) AEROSOL-GENERATING DEVICE FOR FLAT THIN CONSUMABLE

(71) Applicant: Philip Morris Products S.A., Neuchatel (CH)

(72) Inventors: Rui Nuno Rodrigues Alves BATISTA, Morges (CH); Anzhelika SIMONYAN, Yerevan (AM)

(73) Assignee: Philip Morris Products S.A., Neuchatel (CH)

(21) Appl. No.: 19/475,293

(22) PCT Filed: Apr. 18, 2024
(86) PCT No.: PCT/EP2024/060520

§ 371 Date: Oct. 14, 2025

(30) Foreign Application Priority Data
Apr. 21, 2023 (AM) AM20230039

(51) Int. Cl. A24F 40/465; A24F 7/00; A24F 40/20; A24F 40/48; A24F 40/485; H05B 6/02

(52) U.S. Cl. CPC A24F 40/465; A24F 7/00; A24F 40/20; A24F 40/48; A24F 40/485; H05B 6/02

(57) ABSTRACT

An aerosol-generating device is provided, including a main body and a mouthpiece that is configured to be attached to the main body, the mouthpiece having a proximal end defining an outlet opening configured to discharge a generated aerosol, and the mouthpiece having a distal end defining a receptacle configured to receive an aerosol-forming substrate. An aerosol-generating system including the aerosol-generating device and an aerosol-generating article including an aerosol-forming substrate is also provide.

The present invention relates to an aerosol-generating device and an aerosol-generating system.

It is known to provide an aerosol-generating device for generating an inhalable vapor. Such devices may heat aerosol-forming substrate to a temperature at which one or more components of the aerosol-forming substrate are volatilised without burning the aerosol-forming substrate. Aerosol-forming substrate may be provided as part of an aerosol-generating article. The aerosol-generating article may have a rod shape for insertion of the aerosol-generating article into a cavity, such as a heating chamber, of the aerosol-generating device. A heating element may be arranged in or around the heating chamber for heating the aerosol-forming substrate once the aerosol-generating article is inserted into the heating chamber of the aerosol-generating device.

Some aerosol-generating devices are configured to be used with flat, thin consumables, which are heated by planar heating elements. Such arrangements may achieve a more uniform heating of the aerosol-forming substrate contained in the aerosol-generating articles, and higher overall performance in thermal transfer and related aerosolization performance. However, these devices usually require to tightly enclose the aerosol-forming substrate inside the heating cavity, which may affect airflow distribution. In addition, handling of the device, in particular replacing of spent consumables, may be cumbersome.

It would be desirable to have an aerosol-generating device with improved airflow distribution through the consumable. It would be desirable to have an aerosol-generating device with improved heating of an aerosol-forming substrate. It would be desirable to have an aerosol-generating device that can be assembled and disassembled in an easy way. It would be desirable to have an aerosol-generating device with simplified design that facilitates insertion and extraction of consumables. It would be desirable to have an aerosol-generating device providing an optimized user experience.

According to the invention there is provided an aerosol-generating device. The aerosol-generating device may comprise a main body and a mouthpiece that is configured to be attached to the main body. The mouthpiece may have a proximal end defining an outlet opening for discharging a generated aerosol. The mouthpiece may further have a distal end defining a receptacle configured for receiving an aerosol-forming substrate.

According to the invention there is provided an aerosol-generating device. The aerosol-generating device comprises a main body and a mouthpiece that is configured to be attached to the main body. The mouthpiece has a proximal end defining an outlet opening for discharging a generated aerosol. The mouthpiece has a distal end defining a receptacle configured for receiving an aerosol-forming substrate.

By providing a mouthpiece with an integrated receptacle to accommodate an aerosol-forming substrate handling of the aerosol-generating device is simplified. A simplified handling may provide an enhanced user experience.

The receptacle of the mouthpiece may communicate with the outlet opening via an air flow channel. Aerosol generated by heating the aerosol-forming substrate may be conveyed from the receptacle through the air flow channel towards the outlet opening of the mouthpiece. The generated aerosol portion may be discharged via the outlet opening of the mouthpiece.

The mouthpiece may be made from a suitable polymeric material. Such polymeric materials may be selected from low thermal conductivity polymers, high-density polyethylene (HDPE), polypropylene (PP), polystyrene (PS), fluorinated ethylene propylene (FEP), polytetrafluoroethylene (PTFE), polyoxymethylene (POM), epoxy resins, polyurethane resins, vinyl resins, liquid crystal polymers (LCP) and modified LCPs, such as LCPs with graphite or glass fibres.

The receptacle at the distal end of the mouthpiece may be formed from a different material than the remainder of the mouthpiece. The receptacle may be formed from a material that allows inductive heating of the receptacle. The receptacle may be formed from inductively heatable material. The inductively heatable material of the receptacle may be formed from ferromagnetic materials, ferromagnetic alloys, or aluminium containing materials. The inductively heatable ferromagnetic materials may include ferritic iron. The inductively heatable ferromagnetic alloys may include ferromagnetic steel, stainless steel and ferrite.

By forming the receptacle from an inductively heatable material, the receptacle may be used as a susceptor of an inductive heating arrangement. When coupled to the main body of the aerosol-generating device, an induction coil of an inductive heating arrangement arranged within the main body may be configured to inductively heat the receptacle and the aerosol-forming substrate received therein. In this way, an aerosol may be generated than can be inhaled by a user through the outlet opening of the mouthpiece.

The consumable to be received in the receptacle may be a planar consumable. The consumable may be a sheet-like consumable. The consumable may be a pouch-like consumable. The consumable may be formed as block. The consumable may comprise a solid aerosol-forming substrate.

As used herein, the term ‘planar’ relates to an element having a substantially greater length and width than thickness. The length and width directions are orthogonal to one another and define a first plane. The thickness extends orthogonal to the first plane. A planar element may have two opposing major surfaces extending in plane parallel to the first plane. One or both major surfaces are advantageously flat.

The consumable may have a length of between 5 and 50 millimetres. The consumable may have a length of between 10 and 40 millimetres. The consumable may have a length of between 10 and 30 millimetres.

The consumable may have a width of between 5 and 50 millimetres. The consumable may have a width of between 10 and 40 millimetres. The consumable may have a width of between 10 and 30 millimetres.

The consumable may have a thickness of between 0.1 and 10 millimetres. The consumable may have a thickness of between 0.2 and 6 millimetres. The consumable may have a thickness of between 0.5 and 4 millimetres.

The receptacle may have a rectangular cross section. The rectangular cross section of the receptacle may correspond to the dimensions of the planar consumable to be received therein. The receptacle may have width that corresponds to the width of the planar consumable. The receptacle may have height that corresponds to the thickness of the planar consumable. The receptacle may have depth that corresponds to the length of the planar consumable.

The receptacle may have an open distal end. The open distal end of the receptacle may be configured to allow introduction of the consumable comprising aerosol-forming substrate. The receptacle may be formed such that the consumable may snugly fit into the receptacle. The consumable may be retained within the receptacle by friction, only. In such configuration no further retaining means are required for retaining the consumable within the receptacle.

The depth of the receptacle may be smaller than the length of the consumable. Upon full insertion of the consumable into the receptacle, a portion of the consumable may extend from the receptacle. During extraction of a used consumable a user may grab the portion of the consumable that extends outside of the receiving cavity. In this way removal of a used consumable may be facilitated.

The depth of the receptacle may have a size that largely corresponds to the length of the consumable. In this case, a fully inserted consumable may be flush with the open distal end at the rim portion of the receptacle. One of the edges of the rim portion of the receptacle may be configured to comprise a notch. One or both of the edges of the major side surfaces of the receptacle may comprise a notch. The one or more notches may be formed such that they each expose a portion of the lateral surface of an inserted consumable. During extraction of a used consumable a user may grab the portion of the consumable at such exposed portions. In this way the notches may facilitate removal of a used consumable.

The mouthpiece may be configured to provide a thermal insulation between the receptacle and an outer wall of the mouthpiece. The mouthpiece may be configured to incorporate an embedded thermal insulation material, which extends between the receptacle and the outer wall of the mouthpiece. The mouthpiece may be formed from co-extrusion of a thermally insulating plastic and an inductively heatable metal.

The mouthpiece may have a rectangular cross-section. The mouthpiece may have a length of between 10 and 50 millimetres. The mouthpiece may have a length of between 20 and 40 millimetres.

The mouthpiece may have a width of between 10 and 50 millimetres. The mouthpiece may have a width of between 20 and 40 millimetres.

The mouthpiece may have a thickness of between 10 and 50 millimetres. The mouthpiece may have a thickness of between 20 and 40 millimetres.

The main body of the aerosol-generating device may comprise a cavity at its proximal end. The cavity may be configured for receiving the receptacle of the mouthpiece. The cavity may have a cross-section and a depth that correspond to the size of the receptacle of the mouthpiece to be received within the cavity.

The main body and the mouthpiece may comprise corresponding engagement elements. The engagement elements may allow a mechanical engagement between the main body and the mouthpiece. The engagement elements may be configured such as to allow a releasable engagement between the main body and the mouthpiece.

The engagement elements may comprise corresponding threaded portions, snap fit portions or press fit portions. The engagement elements may be formed by the specific shape and dimensions of the receptacle of the mouthpiece and the cavity of the main body. The outer cross-section of the receptacle of the mouthpiece may be formed such as to correspond to the inner cross-section of the cavity of the main body. A friction fit between the receptacle and the cavity may be sufficient to maintain the mouthpiece and the main body in a connected configuration. Such friction fit between the receptacle and the cavity may be particularly advantageous since this allows a simple design that does not require any additionally formed or shaped mechanical components.

A hermetic seal may be provided between the engagement elements of the mouthpiece and the main body. The hermetic seal may comprise a sealing member extending around the full perimeter of the mouthpiece and the main body, respectively. Such sealing member may be obtained from over-molded material around the edge of the receptacle. In another configuration, a sealing member may comprise a sealing gasket. A sealing gasket may be advantageously used with engagement elements comprising threaded portions between the receptacle and the cavity. By using sealing members, the aerosol-generating device may be sealed against air and aerosol leakages. Sealing members may at least reduce the likelihood of leakage of an aerosol-generating device.

The aerosol-generating device may comprise a heating arrangement. The heating arrangement may be an inductive heating arrangement. The inductive heating arrangement may comprise a susceptor and one or more induction coils.

As indicated above, the susceptor of the heating arrangement is formed by the receptacle holding the aerosol-forming substrate.

The one or more induction coils may be provided adjacent the cavity of the main body. The one or more induction coils may be provided to surround the cavity of the main body.

The heating arrangement may comprise one or more flat induction coils. The heating arrangement may comprise a flat induction coil provided on either side of the cavity. The heating arrangement may comprise a flat induction coil provided on either of the major sides of the cavity. Flat heating coils can be provided directly adjacent to surfaces forming the cavity of the mouthpiece. Thus, flat heating coils allow a compact design and allow an efficient heating configuration.

The one or more induction coils may even be arranged within one or more side walls of the mouthpiece forming the receiving cavity. By incorporating the induction coils within the side walls of the cavity, a particularly compact design may be obtained. Further, the side walls may protect the induction coils from inadvertent damage during assembly and use of the aerosol-generating device.

In use of the aerosol-generating device, the cavity may be directly adjacent to the heated receptacle and may therefore be subjected to increased temperatures. It may therefore be advantageous to arrange a thermal insulation to surround the cavity. The thermal insulation may be provided as on or more layers of material that are configured to surround the cavity. The cavity wall may also be completely formed from a thermally insulating material. In such configuration it may be particularly advantageous to arrange the one or more induction coils to be embedded in the cavity walls. By use of the thermal insulation, heat generated within the receptacle may be more efficiently preserved and heat losses through heat conduction may be limited. At the same time the thermal insulation may help to prevent overheating of any outer surfaces of the main body or the mouthpiece of the aerosol-generating device.

The main body of the aerosol-generating device may define an airflow path that extends from an air inlet at the periphery of the main body to the cavity at the proximal end of the main body. Within the main body and upstream from the cavity the distribution chamber may fluidly communicate the cavity with the airflow path. The air flow path may be configured to comprise an air distribution chamber. The air distribution chamber may be provided upstream from the cavity. The air distribution chamber may be provided directly upstream from the cavity. The air distribution chamber may be configured such that the incoming ambient air is more uniformly distributed over the full cross-section of the cavity and the aerosol-forming substrate located in the receptacle, respectively.

A more even air distribution contributes to an effective cooling of the aerosol-forming substrate. A more even air distribution may help to maximize the release of active ingredients from the aerosol-forming substrate. Thereby an aerosol may be generated which results in an increased user satisfaction.

In a simple configuration the air distribution chamber may be defined as a portion of the cavity located at the upstream end of the cavity. The air distribution chamber may be defined as a void portion of the cavity at the upstream end of the cavity which is not occupied by the receptacle and the aerosol-forming substrate, when the mouthpiece is connected to the main body. The air distribution chamber may also be portion between the cavity and the upstream airflow path along which the cross-section of the air flow path expands to the cross-section of the cavity. During use of the aerosol-generating device, the air distribution chamber serves to distribute the air uniformly along the cross section of the substrate such that identical airflow conditions may be obtained essentially over the entire cross-section of the aerosol-forming substrate.

As used herein, the term ‘aerosol-generating device’ refers to a device that interacts with a consumable to generate an aerosol.

As used herein, the term ‘aerosol-generating system’ refers to the combination of an aerosol-generating device with a consumable. In the system, the aerosol-generating device and the consumable cooperate to generate a respirable aerosol.

Preferably, the aerosol-generating device is portable. The aerosol-generating device may have a size comparable to a conventional cigar or cigarette. The device may be an electrically operated smoking device. The device may be a handheld aerosol-generating device. The aerosol-generating device may have a total length between 30 millimetres and 150 millimetres. The aerosol-generating device may have a total length between 86 millimetres and 130 millimetres.

The aerosol-generating device may comprise a housing. The housing may comprise any suitable material or combination of materials. Examples of suitable materials include metals, alloys, ceramics, polymers or composite materials containing one or more of those materials, or thermoplastics that are suitable for food or pharmaceutical applications, for example polypropylene, polyetheretherketone (PEEK) and polyethylene. Preferably, the material is light and non-brittle.

The housing may be elongate. The housing may have a rectangular cross-section.

The aerosol-generating device may have a cylindrical shape. The aerosol-generating device may have an external diameter between 5 millimetres and 30 millimetres.

The housing may comprise at least one air inlet. The housing may comprise more than one air inlet.

Operation of the heating arrangement may be triggered by a puff detection system. Alternatively, the heating arrangement may be triggered by pressing an on-off button, held for the duration of the user's puff. The puff detection system may be provided as a sensor, which may be configured as an airflow sensor to measure the airflow rate. The airflow rate is a parameter characterizing the amount of air that is drawn through the airflow path of the aerosol-generating device per time by the user. The initiation of the puff may be detected by the airflow sensor when the airflow exceeds a predetermined threshold. Initiation may also be detected upon a user activating a button. The sensor may also be configured as a pressure sensor.

The aerosol-generating device may include a user interface to activate the aerosol-generating device, for example a button to initiate heating of the aerosol-generating device or a display to indicate a state of the aerosol-generating device or of the aerosol-forming substrate.

The aerosol-generating device may include additional components, such as, for example a charging unit for recharging an on-board electric power supply in an electrically operated or electric aerosol-generating device.

As used herein, the term ‘proximal’ refers to a user-end, or mouth-end of the aerosol-generating device or system or a part or portion thereof, and the term ‘distal’ refers to the end opposite to the proximal end. When referring to the heating chamber, the term ‘proximal’ refers to the region closest to the open end of the cavity and the term ‘distal’ refers to the region closest to the closed end.

As used herein, the terms ‘upstream’ and ‘downstream’ are used to describe the relative positions of components, or portions of components, of the aerosol-generating device in relation to the direction in which a user draws on the aerosol-generating device during use thereof.

The term ‘airflow path’ as used herein denotes a channel suitable to transport gaseous media. An airflow path may be used to transport ambient air. An airflow path may be used to transport an aerosol. An airflow path may be used to transport a mixture of air and aerosol.

As used herein, a ‘susceptor’ or ‘susceptor element’ means an element that heats up when subjected to an alternating magnetic field. This may be the result of eddy currents induced in the susceptor element, hysteresis losses, or both eddy currents and hysteresis losses. During use, the susceptor element is located in thermal contact or close thermal proximity with an aerosol-forming substrate received in the aerosol-generating device. In this manner, the aerosol-forming substrate is heated by the susceptor such that an aerosol is formed.

The susceptor material may be any material that can be inductively heated to a temperature sufficient to aerosolize an aerosol-forming substrate. Suitable materials for the susceptor material include graphite, molybdenum, silicon carbide, stainless steels, niobium, aluminium, nickel, nickel containing compounds, titanium, and composites of metallic materials. Preferred susceptor materials comprise a metal or carbon. Advantageously the susceptor material may comprise or consists of a ferromagnetic or ferri-magnetic material, for example, ferritic iron, a ferromagnetic alloy, such as ferromagnetic steel or stainless steel, ferromagnetic particles, and ferrite. A suitable susceptor material may be, or comprise, aluminium. The susceptor material may comprise more than 5 percent, preferably more than 20 percent, more preferably more than 50 percent, or more than 90 percent of ferromagnetic, ferri-magnetic or paramagnetic materials. Preferred susceptor materials may be heated to a temperature in excess of 250 degrees Celsius without degradation.

The susceptor material may be formed from a single material layer. The single material layer may be a steel layer.

The susceptor material may comprise a non-metallic core with a metal layer disposed on the non-metallic core. For example, the susceptor material may comprise metallic tracks formed on an outer surface of a ceramic core or substrate.

The susceptor material may be formed from a layer of austenitic steel. One or more layers of stainless steel may be arranged on the layer of austenitic steel. For example, the susceptor material may be formed from a layer of austenitic steel having a layer of stainless steel on each of its upper and lower surfaces. The susceptor element may comprise a single susceptor material. The susceptor element may comprise a first susceptor material and a second susceptor material. The first susceptor material may be disposed in intimate physical contact with the second susceptor material. The first and second susceptor materials may be in intimate contact to form a unitary susceptor. In certain embodiments, the first susceptor material is stainless steel and the second susceptor material is nickel. The susceptor element may have a two-layer construction. The susceptor element may be formed from a stainless-steel layer and a nickel layer.

Intimate contact between the first susceptor material and the second susceptor material may be made by any suitable means. For example, the second susceptor material may be plated, deposited, coated, clad or welded onto the first susceptor material. Preferred methods include electroplating, galvanic plating and cladding.

The aerosol-generating device may comprise a power supply for powering the heating arrangement. The power supply may comprise a battery. The power supply may be a lithium-ion battery. Alternatively, the power supply may be a nickel-metal hydride battery, a nickel cadmium battery, or a lithium-based battery, for example a lithium-cobalt, a lithium-iron-phosphate, lithium titanate or a lithium-polymer battery. The power supply may require recharging and may have a capacity that enables to store enough energy for one or more usage experiences; for example, the power supply may have sufficient capacity to continuously generate aerosol for a period of around six minutes or for a period of a multiple of six minutes. In another example, the power supply may have sufficient capacity to provide a predetermined number of puffs or discrete activations of the heating arrangement.

The power supply may be a direct current (DC) power supply. In one embodiment, the power supply is a DC power supply having a DC supply voltage in the range of 2.5 Volts to 4.5 Volts and a DC supply current in the range of 1 Amp to 10 Amps (corresponding to a DC power supply in the range of 2.5 Watts to 45 Watts). The aerosol-generating device may advantageously comprise a direct current to alternating current (DC/AC) inverter for converting a DC current supplied by the DC power supply to an alternating current. The DC/AC converter may comprise a Class-D, Class-C or Class-E power amplifier. The AC power output of the DC/AC converter is supplied to the induction coil.

The power supply may be adapted to power an inductor coil and may be configured to operate at high frequency. A Class-E power amplifier is preferable for operating at high frequency. As used herein, the term ‘high frequency oscillating current’ means an oscillating current having a frequency of between 500 kilohertz and 30 megahertz. The high frequency oscillating current may have a frequency of from 1 megahertz to 30 megahertz, preferably from 1 megahertz to 10 megahertz, and more preferably from 5 megahertz to 8 megahertz.

In another embodiment the switching frequency of the power amplifier may be in the lower kilohertz range, e.g., between 100 kilohertz and 400 kilohertz. In the embodiments, where a Class-D or Class-C power amplifier is used, switching frequencies in the lower kilohertz range are particularly advantageous.

The aerosol-generating device may comprise a control unit. The control unit may be electrically connected to the one or more inductor coils. The control unit may be configured to control the electrical current supplied to the induction coil(s), and thus the magnetic field strength generated by the induction coil(s).

The power supply and the control unit may be connected to the inductor coil(s).

The control unit may be configured to be able to chop the current supply on the input side of the DC/AC converter. This way the power supplied to the inductor coil(s) may be controlled by conventional methods of duty-cycle management.

Below, there is provided a non-exhaustive list of non-limiting examples. Any one or more of the features of these examples may be combined with any one or more features of another example, embodiment, or aspect described herein.

### Example Ex 1

An aerosol-generating device comprising, a main body and a mouthpiece that is configured to be attached to the main body, wherein the mouthpiece has a proximal end defining an outlet opening for discharging a generated aerosol, and wherein the mouthpiece has a distal end defining a receptacle configured for receiving an aerosol-forming substrate.

### Example Ex 2

The aerosol-generating device according to example 1, wherein the receptacle of the mouthpiece communicates with the outlet opening via an air flow channel.

### Example Ex 3

The aerosol-generating device according to any of the preceding examples, wherein the mouthpiece is made from any of: low thermal conductivity polymers, high-density polyethylene (HDPE), polypropylene (PP), polystyrene (PS), fluorinated ethylene propylene (FEP), polytetrafluoroethylene (PTFE), polyoxymethylene (POM), epoxy resins, polyurethane resins, vinyl resins, liquid crystal polymers (LCP) and modified LCPs, such as LCPs with graphite or glass fibres.

### Example Ex 4

The aerosol-generating device according to any of the preceding examples, wherein the receptacle is formed from inductively heatable material.

### Example Ex 5

The aerosol-generating device according to example 4, wherein the receptacle is formed from any of: ferromagnetic material, for example ferritic iron, ferromagnetic alloy, such as ferromagnetic steel or stainless steel, ferrite and aluminium containing material.

### Example Ex 6

The aerosol-generating device according to any preceding example, wherein a thermal insulation is provided between the receptacle and the outer wall of the mouthpiece.

### Example Ex 7

The aerosol-generating device according to any preceding example, wherein the receptacle has a rectangular cross section.

### Example Ex 8

The aerosol-generating device according to any preceding example, wherein a rim portion of the receptacle comprises a notch.

### Example Ex 9

The aerosol-generating device according to any preceding example, wherein the mouthpiece has a rectangular cross-section.

### Example Ex 10

The aerosol-generating device according to any preceding example, wherein the mouthpiece has a length, width and thickness of between 10 and 50 millimetres, preferably of between 20 and 40 millimetres.

### Example Ex 11

The aerosol-generating device according to any preceding example, wherein the main body and the mouthpiece comprise corresponding engagement elements.

### Example Ex 12

The aerosol-generating device according to example 11, wherein the engagement elements comprise threaded portions, snap fit portions or press fit portions.

### Example Ex 13

The aerosol-generating device according to example 11, wherein the main body comprises a receiving cavity at the proximal end, configured for receiving the receptacle of the mouthpiece.

### Example Ex 14

The aerosol-generating device according to any preceding example, wherein a hermetic seal is provided between the engagement elements of the mouthpiece and the main body.

### Example Ex 15

The aerosol-generating device according to the preceding example, wherein the hermetic seal comprises over-molded material or a sealing gasket.

### Example Ex 16

The aerosol-generating device according to the preceding example, further comprising a heating arrangement for heating the aerosol-forming substrate.

### Example Ex 17

The aerosol-generating device according to the preceding example, wherein the heating arrangement is configured to inductively heat the aerosol-forming substrate.

### Example Ex 18

The aerosol-generating device according to the preceding example, wherein the heating arrangement comprises a flat induction coil.

### Example Ex 19

The aerosol-generating device according to the preceding example, wherein the induction coil is arranged within a side wall of the receiving cavity.

### Example Ex 20

The aerosol-generating device according to the preceding example, wherein the induction coil surrounds the receiving cavity.

### Example Ex 21

The aerosol-generating device according to the preceding example, wherein the induction coil is arranged on each side of the cavity.

### Example Ex 22

The aerosol-generating device according to the preceding example, wherein a thermal insulation layer is arranged to surround the cavity.

### Example Ex 23

The aerosol-generating device according to the preceding example, wherein an air distribution chamber is provided upstream from the cavity.

### Example Ex 24

The aerosol-generating device according to the preceding example, wherein the air distribution chamber is connected to an air inlet on the periphery of the main body.

### Example Ex 25

The aerosol-generating device according to the preceding example, wherein the air distribution chamber is configured to distribute the air uniformly along the cross section of the substrate.

### Example Ex 26

The aerosol-generating device according to the preceding example, wherein the main body comprises a housing, and wherein the housing is made from metal, metal alloys, ceramics, polymers, composite material, or a combination thereof.

### Example Ex 27

The aerosol-generating device according to the preceding example, wherein the housing has a length of between 50 and 150 millimetres, preferably of between 86 to 130 millimetres.

### Example Ex 28

The aerosol-generating device according to the preceding example, wherein the main body comprises a power supply and a control unit, wherein the control unit is configured to control a supply of electric power from the power supply to the heating arrangement.

### Example Ex 29

The aerosol-generating system comprising an aerosol-generating device to any of the preceding examples and an aerosol-generating article comprising an aerosol-forming substrate.

### Example Ex 30

The aerosol-generating system according to the preceding example, wherein the receptacle of the mouthpiece and the aerosol-generating article have a corresponding cross-section, preferably a corresponding rectangular cross-section.

### Example Ex 31

The aerosol-generating system according to the preceding example, wherein the aerosol-generating article comprises an aerosol-forming substrate comprising solid tobacco material, for example shredded tobacco or a homogenized tobacco material with a porous structure.

### Example Ex 32

The aerosol-generating system according to the preceding example, wherein the aerosol-generating article has a length of between 10 and to 30 millimetres, a width of between 10 and 30 millimetres and a thickness of between 0.2 to 6 millimetres, preferably from 0.5 to 4 millimetres.

Features described in relation to one embodiment may equally be applied to other embodiments of the invention.

The invention will be further described, by way of example only, with reference to the accompanying drawings in which:

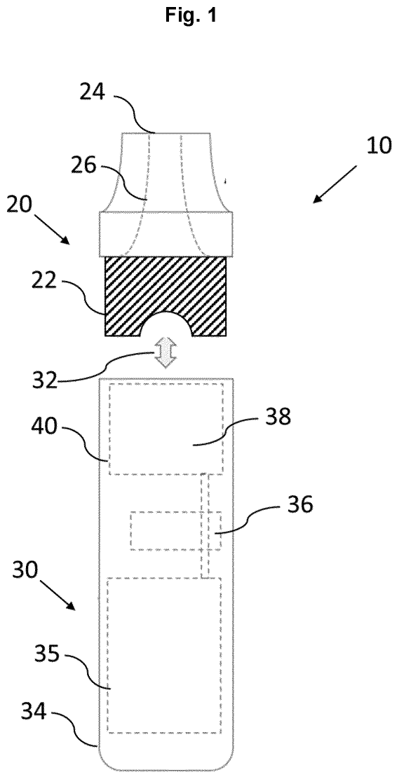

FIG. 1 shows an aerosol-generating device comprising a mouthpiece and main body;

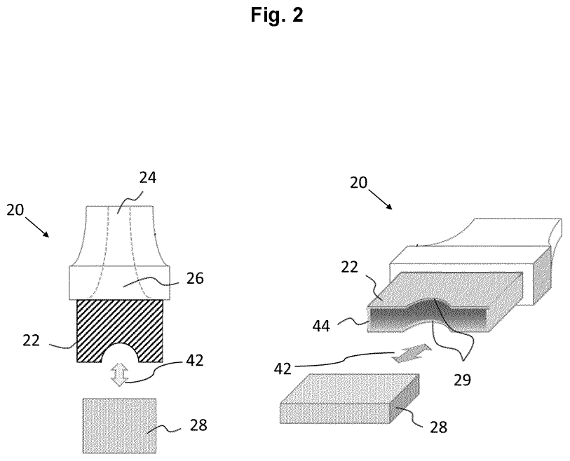

FIG. 2 shows a mouthpiece configured for receiving an aerosol-forming substrate;

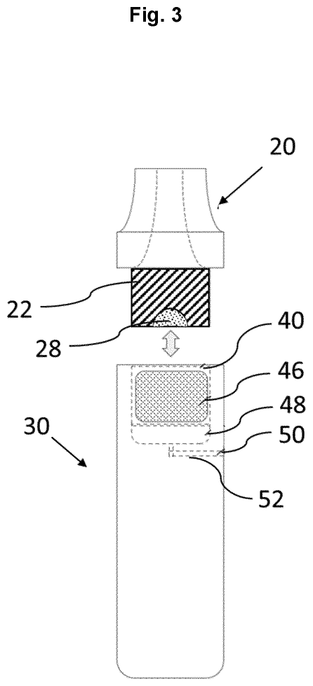

FIG. 3 shows details of the airflow path through the main portion of an aerosol-generating device; and

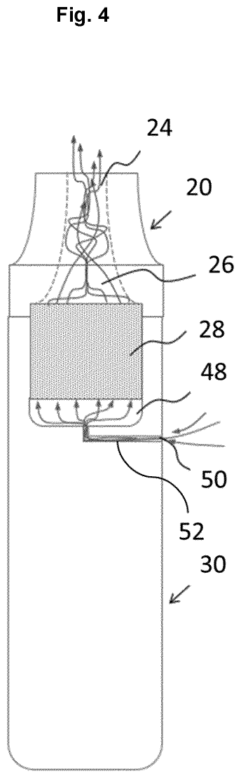

FIG. 4 shows a fully assembled aerosol-generating device.

FIG. 1 shows an aerosol-generating device 10 comprising a mouthpiece 20 of and a main body 30 in a detached configuration.

The mouthpiece 20 has a distal end defining a receptacle 22 configured for receiving a consumable comprising an aerosol-forming substrate. The receptacle 22 is formed from ferromagnetic steel. The receptacle 22 forms a first part of the inductive heating arrangement of the aerosol-generating device 10. In more detail, the receptacle 22 acts as a susceptor that may be inductively heated. The mouthpiece 20 has a proximal end defining an outlet opening 24 for discharging a generated aerosol. The outlet opening 24 is in fluid communication with the receptacle 22 via airflow channel 26.

The mouthpiece 20 is releasably attachable to the main body 30. An arrow 32 indicates the direction of attaching the mouthpiece 20 to the main body 30. The main body 30 has an elongated housing 34 with a cavity 40 formed at its proximal end. The cavity is configured to receive the receptacle, when the mouthpiece 20 is attachable to the main body 30.

The housing of the main body 30 comprises a power supply 35, and a control unit 36 configured to control a supply of electric power from the power supply 35 to an induction coil 38. The induction coil 38 is located adjacent to the cavity 40 and forms a second part of the heating arrangement of the aerosol-generating device 10.

When the mouthpiece 20 is attached to the main body 30, the induction coil 38 is located in direct vicinity of the receptacle 22. The control unit 36 may control the induction coil 38 to generate an alternating magnetic field, which heats up the susceptor material of the receptacle 22. In this way an aerosol-forming substrate comprised in the receptacle 22 may be heated to release its volatile components.

FIG. 2 shows a side view and a perspective view of the mouthpiece of FIG. 1 and its intended use with a flat, planar consumable 28. The consumable 28 is a rectangular shaped block of aerosol-generating substrate. The consumable 28 has a width of 30 millimetres, a length of 25 millimetres and a thickness of 1 millimetres. As indicated by arrows 42, the consumable 28 is to be inserted into the open distal end 44 of the rectangular receptacle 22. The cross-section of the receptacle 22 corresponds to the cross-section of the consumable 28. A form-fit between the receptacle 22 and the consumable 28 is sufficient to retain the inserted consumable 28 within the receptacle 22.

At the rim portion of the distal end 44 of the receptacle 22, two notches 29 are formed. The notches 29 are formed at the opposing major edges of the receptacle 22. The notches 29 expose a portion of an inserted consumable 28. The exposed portions may be gripped by a user and may thereby facilitate removal of the consumable 28.

FIG. 3 shows further details of the aerosol-generating device 10. The mouthpiece 20 has a receptacle 22 at its distal end. In the embodiment depicted in FIG. 3, a consumable 28 is already inserted into the receptacle 22. The distal end of the consumable 28 lies flush with the rim portion of the receptacle 22.

Before use, the mouthpiece 20 with the inserted consumable 28 is to be attached to the main body 30. To this end the mouthpiece 20 is mechanically engaged with the main body 30. The distal end of the mouthpiece 20 and the proximal end of the main body 30 have corresponding engagement elements. In the embodiment of FIG. 3, the engagement elements are formed by the specific shape and dimensions of the receptacle 22 of the mouthpiece 20 and the cavity 40 of the main body 30. In more detail, the receptacle 22 has an outer cross-section that corresponds to the inner cross-section of the cavity 40 and both components form a friction fit connection. Such friction fit connection allows a particularly simple design of the aerosol-generating device 10.

In the embodiment of FIG. 3, two flat induction coils 46 are integrate in the wall portion forming the cavity 40 of the main body 30. At the distal end of the cavity 40 there is provided a hollow space that forms an air distribution chamber 48. The air distribution chamber 48 is communicated with an air inlet 50 via an air inlet channel 52. The air distribution chamber 48 assists in providing a uniform airflow distribution over the complete cross-section of the aerosol-forming substrate of the consumable 28.

FIG. 4 shows an assembled aerosol-generating device 10. In particular, FIG. 4 indicates the airflow within an aerosol-generating device 10 in use. When a user takes a puff, an airflow through the aerosol-generating device is established. This airflow may be sensed by an airflow sensor (not shown) which activates the heating arrangement.

The arrows in FIG. 4 schematically depict the airflow through the aerosol-generating device 10. Fresh ambient air enters into the air flow channel 52 at air inlet 50. The airflow is communicated to the air distribution chamber 48. In the air distribution chamber 48 the cross-section of the airflow expands to the size of the cross-section of the inserted consumable 28. In the air distribution chamber 48 the airflow is distributed uniformly before traveling upwards through the aerosol-forming substrate of the consumable 28. The airflow travels through the aerosol-forming substrate and entrains volatilized particles that are released from the heated aerosol-forming substrate. Thereby an aerosol is formed. The aerosol exits the receptacle 22 towards the outlet opening 24 via airflow channel 26 within the mouthpiece 10.

After consumption of the active ingredients of the aerosol-forming substrate, the consumable 28 may be exchanged. To this end the mouthpiece 20 may be pulled to detach it from the main body 30 of the aerosol-generating device 10. The spent consumable 28 may be removed from the receptacle 22, and then be replaced by a fresh consumable 28.

1.-15. (canceled)

16. An aerosol-generating device, comprising:
a main body and a mouthpiece that is configured to be attached to the main body,
wherein the mouthpiece has a proximal end defining an outlet opening configured to discharge a generated aerosol, and
wherein the mouthpiece has a distal end defining a receptacle configured to receive an aerosol-forming substrate.

17. The aerosol-generating device according to claim 16, wherein the receptacle is formed from inductively heatable material.

18. The aerosol-generating device according to claim 16, wherein a thermal insulation is provided between the receptacle and an outer wall of the mouthpiece.

19. The aerosol-generating device according to claim 16, wherein the receptacle has a rectangular cross section.

20. The aerosol-generating device according to claim 16, wherein a rim portion of the receptacle comprises a notch.

21. The aerosol-generating device according to claim 16, wherein the main body and the mouthpiece comprise corresponding engagement elements.

22. The aerosol-generating device according to claim 16, wherein the main body comprises a receiving cavity at the proximal end, configured to receive the receptacle.

23. The aerosol-generating device according to claim 21, wherein a hermetic seal is provided between the engagement elements of the mouthpiece and the main body.

24. The aerosol-generating device according to claim 23, further comprising a heating arrangement configured to heat the aerosol-forming substrate.

25. The aerosol-generating device according to claim 24, wherein the heating arrangement is configured to inductively heat the aerosol-forming substrate.

26. The aerosol-generating device according to claim 25, wherein a thermal insulation layer is arranged to surround the cavity.

27. The aerosol-generating device according to claim 26, wherein an air distribution chamber is provided upstream from the cavity.

28. The aerosol-generating device according to claim 27, wherein the air distribution chamber is configured to distribute the air uniformly along a cross section of the aerosol-forming substrate.

29. An aerosol-generating system comprising the aerosol-generating device according to claim 16 and an aerosol-generating article comprising an aerosol-forming substrate.

30. The aerosol-generating system according to claim 29, wherein the receptacle of the mouthpiece and the aerosol-generating article have a corresponding cross-section.

31. The aerosol-generating system according to claim 29, wherein the receptacle of the mouthpiece and the aerosol-generating article have a corresponding rectangular cross-section.
